(19) United States
(12) Patent Application Publication
(10) Pub. No.: US 2014/0299133 A1
(43) Pub. Date: Oct. 9, 2014

(54) HOSE MANAGEMENT SYSTEM FOR A RESPIRATORY THERAPY DEVICE

(71) Applicant: KONINKLIJKE PHILIPS N.V., EINDHOVEN (NL)

(72) Inventors: Amanda Marie Neely, Monroville, PA (US); Richard Thomas Haibach, Verona, PA (US); John Theodore Gobble, III, Pittsburgh, PA (US); Robert William Baiko, Pittsburgh, PA (US)

(21) Appl. No.: 14/351,338

(22) PCT Filed: Oct. 8, 2012
(86) PCT No.: PCT/IB2012/055416

§ 371 Date: Apr. 11, 2014

Related U.S. Application Data
(60) Provisional application No. 61/549,966, filed on Oct. 21, 2011.

(51) Int. Cl. A61M 16/08; A61M 16/06; A61M 16/00; B65H 75/44

(52) U.S. Cl. CPC A61M 16/0875; B65H 75/4471; A61M 16/06; A61M 16/0057
USPC 128/205.25; 242/398

(57) ABSTRACT

A hose management system (30) for use with a system adapted to provide respiratory therapy. The system includes an elongated, flexible second hose (22). The hose management system (30) includes a housing assembly (32), a rotatable spool assembly (34), and a rotatable coupling (36). The housing assembly has a sidewall assembly (40) defining a substantially enclosed space. The rotatable spool assembly has a spool body (50). The spool body (50) is disposed in the housing assembly (32) enclosed space and rotatably coupled to the housing assembly (32). The rotatable coupling (36) defines a passage (90), the rotatable coupling (36) being a rotatable conduit extending through the housing assembly sidewall and spool body. Wherein the second hose (22) may be substantially wrapped about the spool body (50) and substantially concealed within the housing assembly (32) enclosed space.

### CROSS-REFERENCE TO RELATED APPLICATIONS

This patent application claims the priority benefit under 35 U.S.C. §119(e) of U.S. Provisional Application No. 61/549,966 filed on Oct. 21, 2011, the contents of which are herein incorporated by reference.

### BACKGROUND OF THE INVENTION

1. Field of the Invention

The present invention pertains to a hose management system and, in particular, to a hose management system for a medical device wherein the hose management system substantially conceals a hose when the hose is not in use.

2. Description of the Related Art

There are numerous situations where it is necessary or desirable to deliver a flow of breathing gas non-invasively to the airway of a patient, i.e., without intubating the patient or surgically inserting a tracheal tube in their esophagus. For example, it is known to ventilate a patient using a technique known as non-invasive ventilation. It is also known to deliver continuous positive airway pressure (CPAP) or variable airway pressure, which varies with the patient's respiratory cycle, to treat a medical disorder, such as sleep apnea syndrome, in particular, obstructive sleep apnea (OSA), or congestive heart failure.

Non-invasive ventilation and pressure support therapies utilize a ventilator or pressure support device and a patient interface device, typically a mask component, that are coupled and in fluid communication via a delivery conduit, typically a hose. The mask component may be, without limitation, a nasal mask that covers the patient's nose, a nasal cushion having nasal prongs that are received within the patient's nares, a nasal/oral mask that covers the nose and mouth, or a full face mask that covers the patient's face. The patient interface device interfaces the ventilator or pressure support device with the airway of the patient, so that a flow of breathing gas can be delivered from the pressure/flow generating device to the airway of the patient.

The mask is coupled to the ventilator or pressure support device via a hose that provides fluid communication therebetween. The hose is flexible and typically has a diameter of between about 17 mm and 23 mm, and a length of about six feet. This size allows for a sufficient fluid flow through the hose and a sufficient length to allow the user to move a limited distance. That is, the system is often utilized when a user is sleeping and a six foot hose is of a sufficient length to allow the user to move about during sleep.

The problem with this system is that many users, or patients, have a negative response to the appearance of the system. That is, some people have an aversion to medical equipment as the mere presence of medical equipment implies that the user is not healthy. This aversion may be exacerbated by continually viewing the system. Such an aversion may affect the user's desire to utilize the system. To that end, many users attempt to hide the system from sight, e.g. positioning the system under their bed or on the floor beside their bed. This, however, creates additional problems.

The floor is typically not a clean environment. Thus, the mask is prone to contamination. Further, even when the ventilator or pressure support device is placed on the floor, the user may hang the hose and mask on a bedpost or other elevated location so as to keep the mask clean. This, however, reintroduces the problem of a portion of the system being visible and causing a negative response in the user.

### SUMMARY OF THE INVENTION

Accordingly, it is an object of this invention to provide a hose management system for use with a system adapted to provide a regimen of respiratory therapy for a patient, the system including a pressure generating system adapted to produce a flow of gas, a conduit assembly, and a patient interface, wherein the hose management system encloses, or substantially conceals, the conduit assembly. The conduit assembly is disposed between and in fluid communication with both the pressure generating system and the patient interface. The flexible conduit assembly includes at least a first hose and an elongated, flexible second hose. The hose management system includes a housing assembly having a sidewall assembly that defines a substantially enclosed space, a rotatable spool assembly having a spool body with a generally cylindrical outer surface, and a rotatable coupling. The spool body is disposed in the housing assembly enclosed space and rotatably coupled to the housing assembly. The rotatable coupling defines a passage, the rotatable coupling being a rotatable conduit extending through the housing assembly sidewall and spool body. In this configuration, the conduit assembly second hose may be substantially wrapped about the spool body and substantially disposed within the housing assembly enclosed space.

It is a further object of this invention to provide a method of using a hose management system wherein the hose management system is used in conjunction with a system adapted to provide a regimen of respiratory therapy for a patient, the system includes a pressure generating system adapted to produce a flow of gas, a conduit assembly, and a patient interface. The conduit assembly is disposed between and in fluid communication with both the pressure generating system and the patient interface. The flexible conduit assembly includes at least a first hose and an elongated, flexible second hose. The hose management system includes a housing assembly having a sidewall assembly defining a substantially enclosed space, a rotatable spool assembly having a spool body with a generally cylindrical outer surface, and a rotatable coupling. The spool body is disposed in the housing assembly enclosed space and rotatably coupled to the housing assembly. The rotatable coupling defines a passage having a first upstream end and a second downstream end, the rotatable coupling being a rotatable conduit extending through the housing assembly sidewall and spool body. The first hose is coupled to, and in fluid communication with, the rotatable coupling first end, and the second hose coupled to the rotatable coupling second end. Thus, the conduit assembly second hose may be substantially wrapped about the spool body and substantially disposed within the housing assembly enclosed space. The method of using the hose management system includes the steps of: moving at least a length of the second hose out of the housing assembly, using the second hose to provide a flow of gas to a patient for a treatment, and, after the patient has completed the treatment, moving the length of the second hose that is out of the housing assembly back into the housing assembly.

These and other objects, features, and characteristics of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention.

### BRIEF DESCRIPTION OF THE DRAWINGS

FIG. 1 is an isometric view of a system adapted to provide a regimen of respiratory therapy adjacent to a bed.

FIG. 2 is an isometric view of a hose management system.

FIG. 3 is isometric view of a hose management system with an alternate shape.

FIG. 4 is a cross-sectional side view of a hose management system.

FIG. 5 is a cross-sectional end view of a hose management system.

FIG. 6 is a cross-sectional end view of selected elements of a hose management system.

FIG. 7 is a cross-sectional end view of selected elements of another embodiment of a hose management system.

FIG. 8 is an isometric view of a hose management system.

FIG. 9 is a detail isometric view of a crank.

FIG. 10 is a cross-sectional end view of another embodiment of a crank.

FIG. 11 is a detail isometric view of an automatic hose retraction device.

FIG. 12 is cross-sectional end view of a hose management system with a hose guide device.

FIG. 13 is a flow chart of the steps associated with a method of using a hose management system.

FIG. 14 is a cross-sectional side view of another embodiment.

### DETAILED DESCRIPTION OF THE EXEMPLARY EMBODIMENTS

As used herein, the singular form of “a”, “an”, and “the” include plural references unless the context clearly dictates otherwise. As used herein, the statement that two or more parts or components are “coupled shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other.

As used herein, the word “unitary” means a component is created as a single piece or unit. That is, a component that includes pieces that are created separately and then coupled together as a unit is not a “unitary” component or body. As employed herein, the statement that two or more parts or components “engage” one another shall means that the parts exert a force against one another either directly or through one or more intermediate parts or components. As employed herein, the term “number” shall mean one or an integer greater than one (i.e., a plurality).

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

As used herein, “substantially conceal(ed)” means that an object is substantially enclosed within a housing and cannot be seen from any angle. For example, a hose disposed in a decorative hose reel having openings cut into decorative patterns is not “substantially concealed.” Further, a hose reel having an opening that is substantially larger than the hose does not “substantially conceal” the hose. For example, a hose reel having a slot, as opposed to a circular opening sized to correspond to the size of the hose, does not “substantially conceal” a hose.

As used herein, a “rotatable conduit” is a conduit assembly having at least two elements that are rotatable coupled to each other and wherein the ends of the conduit may rotate independently. That is, the conduit assembly does not twist or otherwise become constrained even if rotated infinitely in one direction.

As used herein, a “ratchet gear” is a gear having teeth that structured to be engaged by a pawl. The teeth may extend inwardly or outwardly from a generally circular gear body. The teeth may be generally radial or may be angled toward a selected direction.

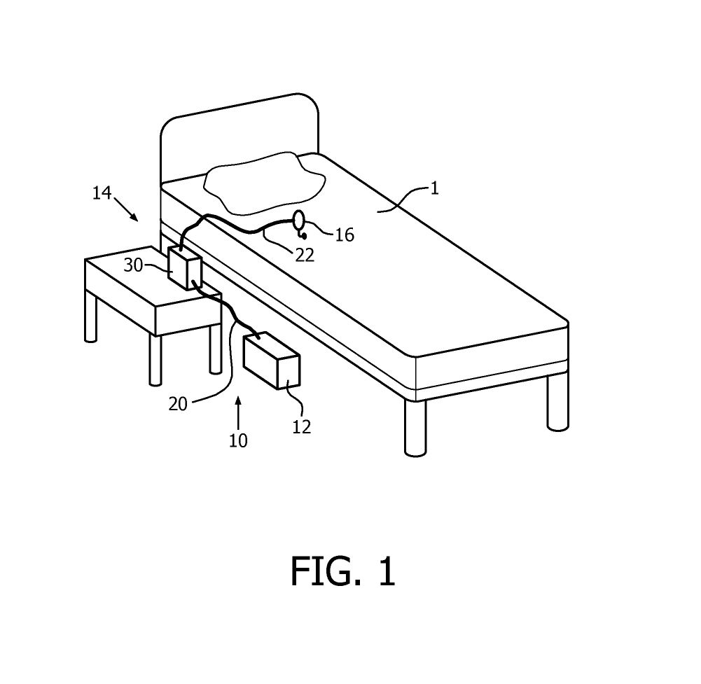

As shown in FIG. 1, a system 10 adapted to provide a regimen of respiratory therapy for a patient includes a pressure generating system 12 adapted to produce a flow of gas, a conduit assembly 14, and a patient interface 16, such as, without limitation, a nasal mask, a nasal cushion, a nasal/oral mask, or a full face mask. Conduit assembly 14 is disposed between, and is in fluid communication with, both pressure generating system 12 and patient interface 16. Thus, a fluid may be transferred from pressure generating system 12 to patient interface 16 via conduit assembly 14. Flexible conduit assembly 14 includes at least a first passage 19 and an elongated, flexible second hose 22. The first passage 19 may be a conduit formed into a hose management system housing assembly 32, discussed below, that is coupled to an outlet on pressure generating system 12. That is, hose management system 30, discussed below, may be coupled directly to pressure generating system 12. In an alternate embodiment, first passage 19 is a first hose 20 extending between, and providing fluid communication between, hose management system 30 and pressure generating system 12. In an exemplary embodiment, second hose 22, has an outer diameter of between about 17 mm and 23 mm, and more preferably of about 22 mm. Second hose 22 preferably has a length of between about 4 feet and 8 feet, and more preferably of about 6 feet.

In one exemplary embodiment, pressure generating system 12 is disposed in an unobtrusive location such as, but not limited to, the floor beside a user's bed 1. First hose 20 extends between, and provides fluid communication between, pressure generating system 12 and hose management system 30. Second hose 22 extends between, and provides fluid communication between, hose management system 30 and patient interface 16. A hose management system 30 is structured to enclose, or substantially conceal, second hose 22 when system 10 is not in use.

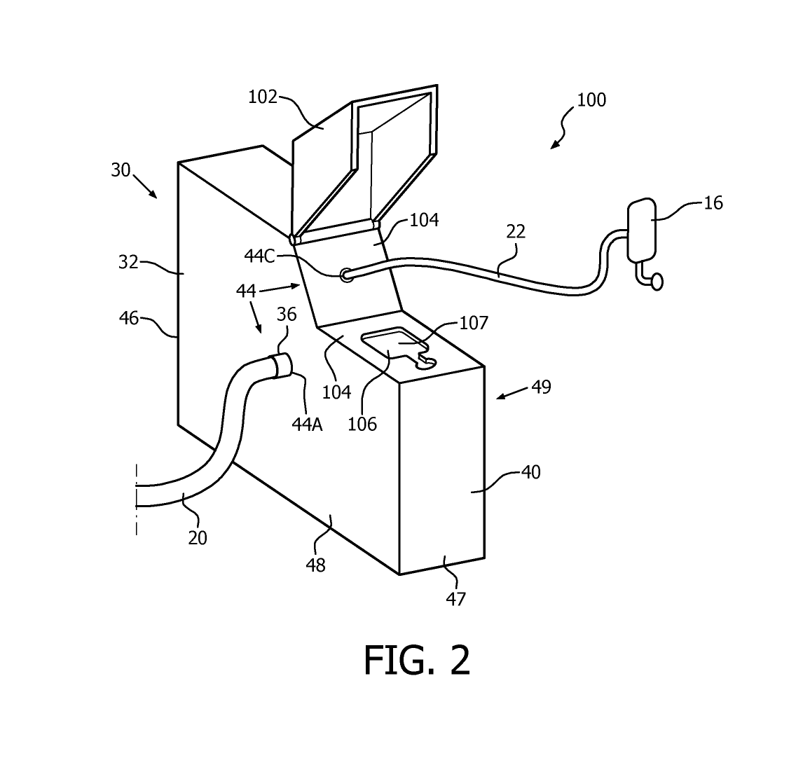

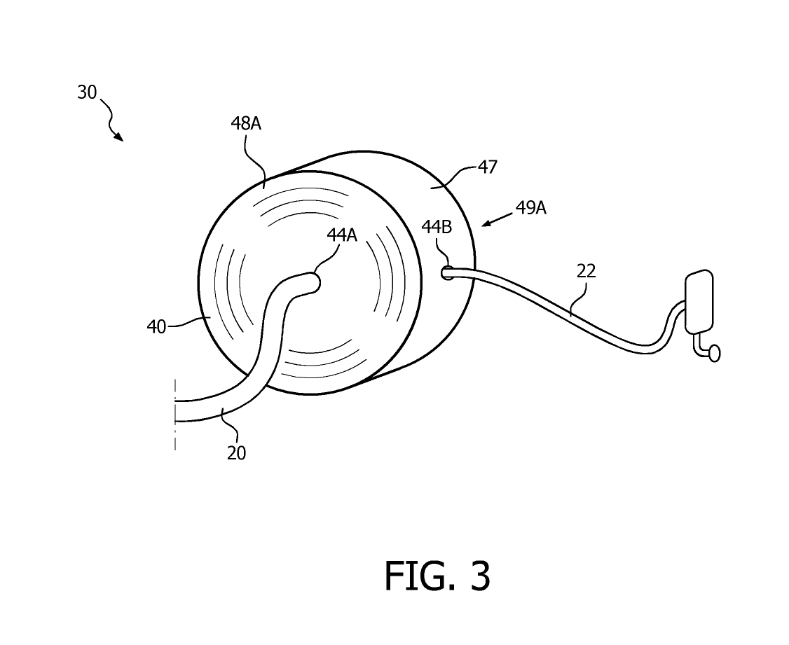

As shown in FIG. 2, hose management system 30 includes a housing assembly 32, a rotatable spool assembly 34 (FIG. 3), and a rotatable coupling 36. Housing assembly 32 includes a sidewall assembly 40 defining a substantially enclosed space 42 (FIG. 3). The present invention contemplates that sidewall assembly 40 is made from molded plastic. The present invention contemplates that housing assembly sidewall assembly 40 has a limited number of openings therethrough. That is, housing assembly sidewall assembly 40 includes three or less openings 44, each opening 44 through which hose 20, 22 passes being substantially the same size as hose 20, 22 passing therethrough. Openings 44 include at least an inlet opening 44A, and an outlet opening 44B (FIG. 3). In this configuration, housing assembly sidewall assembly 40 is a shell 46 that substantially conceals any object in housing assembly enclosed space 42. That is, when hose management system 30 is assembled and hoses 20, 22 extend through at least two openings 44, housing assembly 32 substantially conceals second hose 22 within enclosed space 42. This configuration means that when second hose 22 is disposed substantially within enclosed space 42 it, essentially, cannot be seen and this is one object of the invention.

The present invention contemplates that housing assembly sidewall assembly 40 has a rectangular (FIG. 2) or circular (FIG. 3) box-like shape. That is, housing assembly sidewall assembly 40 includes a first substantially planar member 48 and a second substantially planar member 49. First and second planar members 48, 49 are disposed substantially parallel to each other. First and second planar members 48, 49 may be any shape, but rectangular or circular are preferred. A depending sidewall 47 extends between first and second planar members 48, 49. The present invention contemplates that depending sidewall 47 is disposed at the perimeter of first and second planar members 48, 49. If a spool body 50 (discussed below) is elongated, outlet opening 44B is disposed on depending sidewall 47 aligned with the medial portion of spool body 50. That is, outlet opening 44B is not aligned with the ends of elongated spool body 50.

In the exemplary embodiment, housing assembly sidewall assembly 40 encloses a volume of about 250 in.³, e.g. having dimensions of about 4 inches by 8 inches by 8 inches, which is sufficient to accommodate second hose 22, as described above. If, however, second hose 22 has a greater length than identified above, the volume of enclosed space 42 may be increased by: (1) increasing the area of first and second planar members 48, 49; (2) increasing the spacing between first and second planar members 48, 49, i.e. increasing the height of depending sidewall 47; and/or (3) using arcuate sidewall members 48A, 49A as shown in FIG. 3. Depending upon the volume needed, arcuate sidewall members 48A, 49A may be slightly arcuate, for a small increase in volume, or spherical, for a greater increase in volume.

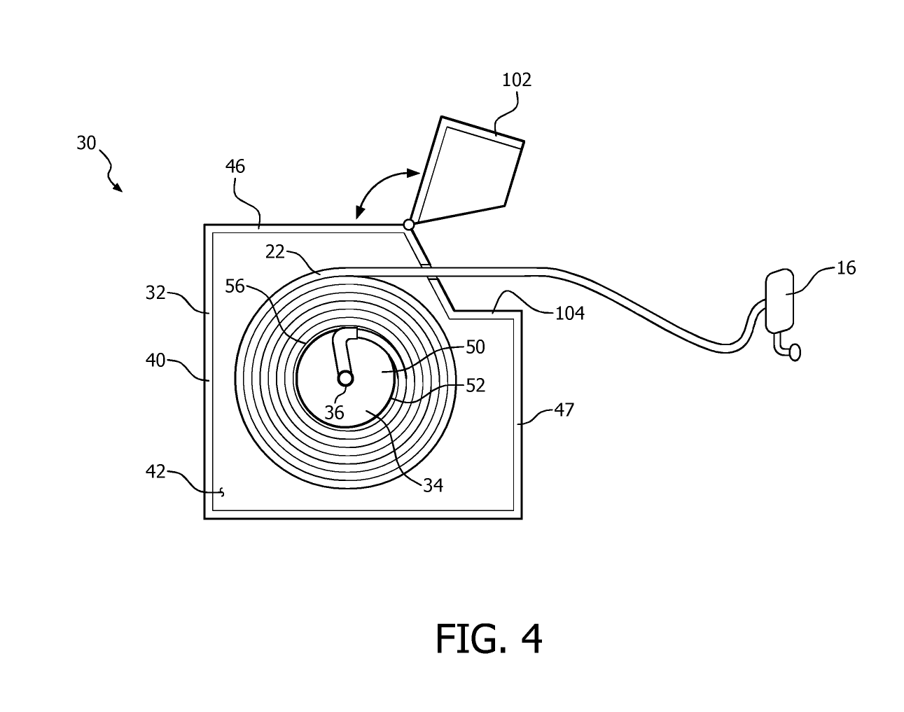

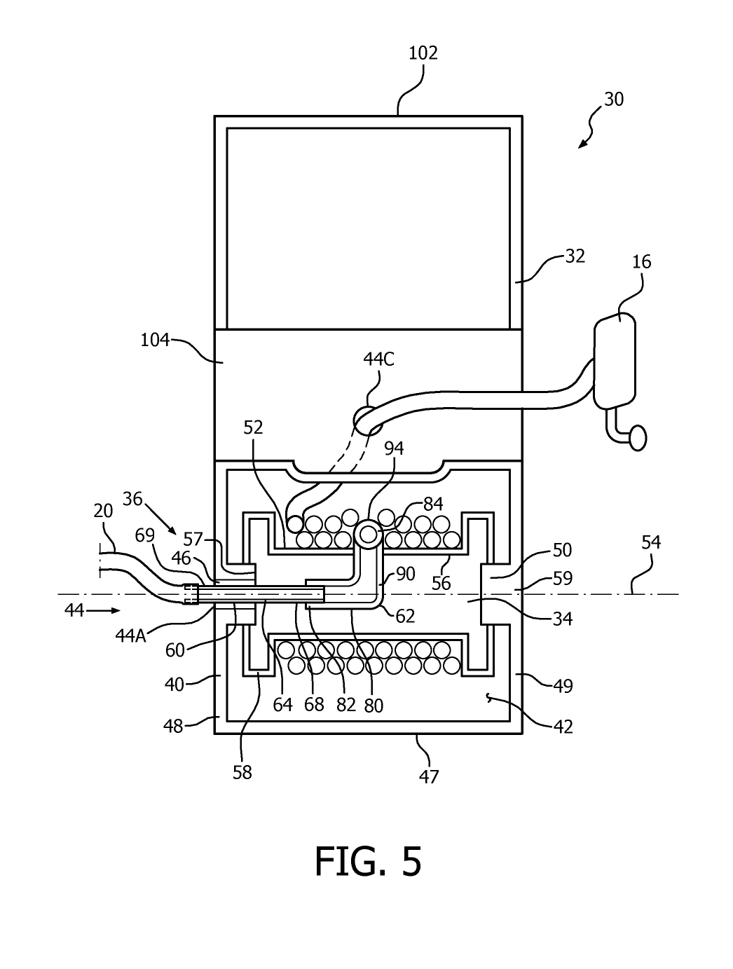

As shown in FIGS. 4 and 5, rotatable spool assembly 34 includes spool body 50 with a generally cylindrical outer surface 52 and an axis of rotation 54. Spool body 50 is a generally cylindrical member. Spool body 50 may be elongated, i.e. have an axis with a similar or greater length than the radius, or, spool body 50 may be a disk, i.e. have a radius that is a much greater length than the axis. The present invention contemplates that spool body 50 includes a barrel 56 and two hubs 58. Barrel 56 has radius that is smaller than hubs 58. Spool body 50 is hollow so as to accommodate separate rotatable couplings 36. Alternatively, spool body 50 may be substantially solid with a passage therethrough defining a portion of rotatable coupling 36, as discussed below. Spool body 50 is sized to fit within enclosed space 42 and is further structured to be rotatably coupled to housing assembly 32. For example, as shown in FIG. 5, outer axial surface of the hubs 58 may include a circular cavity 57. Housing assembly sidewall assembly 40 may include circular projections 59 disposed in housing assembly sidewall assembly 40 inner surface. Circular projections 59 are sized to fit closely within circular cavities 57.

Rotatable coupling 36 is a rotatable conduit extending through housing assembly sidewall assembly 40 and spool body 50. Rotatable coupling 36 allows for fluid communication from one end to the other. The present invention contemplates that rotatable coupling 36 includes two elements; a stationary first conduit 60 and a movable second conduit 62. Stationary first conduit 60 is coupled to, and extends through housing assembly sidewall assembly 40. Stationary first conduit 60 is a tubular body 64 having a first end 66 and a second end 68. The present invention contemplates that stationary first conduit body 64 is a substantially straight hollow circular body 64. A stationary first conduit first end 66 is an inlet and is structured to be coupled to, and in fluid communication with, passage 19, i.e. a direct connection to pressure generating system 12 or first hose 20. Stationary first conduit second end 68 is an outlet structured to be coupled to, and in fluid communication with, movable second conduit 62. Stationary first conduit 60 is identified as “stationary” because first and second ends 66, 68 are maintained in a substantially constant position relative to housing assembly sidewall assembly 40. That is, stationary first conduit 60 extends through housing assembly sidewall assembly 40 and enters spool body 50 substantially on the spool body axis of rotation 54. Stationary first conduit 60 may, however, rotate relative to housing assembly sidewall assembly 40 while still being “stationary.”

In one embodiment, stationary first conduit 60 is a separate element from housing assembly sidewall assembly 40. In this embodiment, stationary first conduit tubular body 64 may be rotatably coupled to housing assembly sidewall assembly 40. In this configuration, housing assembly sidewall assembly 40 may include a bearing 69, or similar element which protects and/or supports housing assembly sidewall assembly 40. Such a bearing 69 may be unitary or separate from housing assembly sidewall assembly 40. However, The present invention contemplates that stationary first conduit tubular body 64 is fixed to housing assembly sidewall assembly 40.

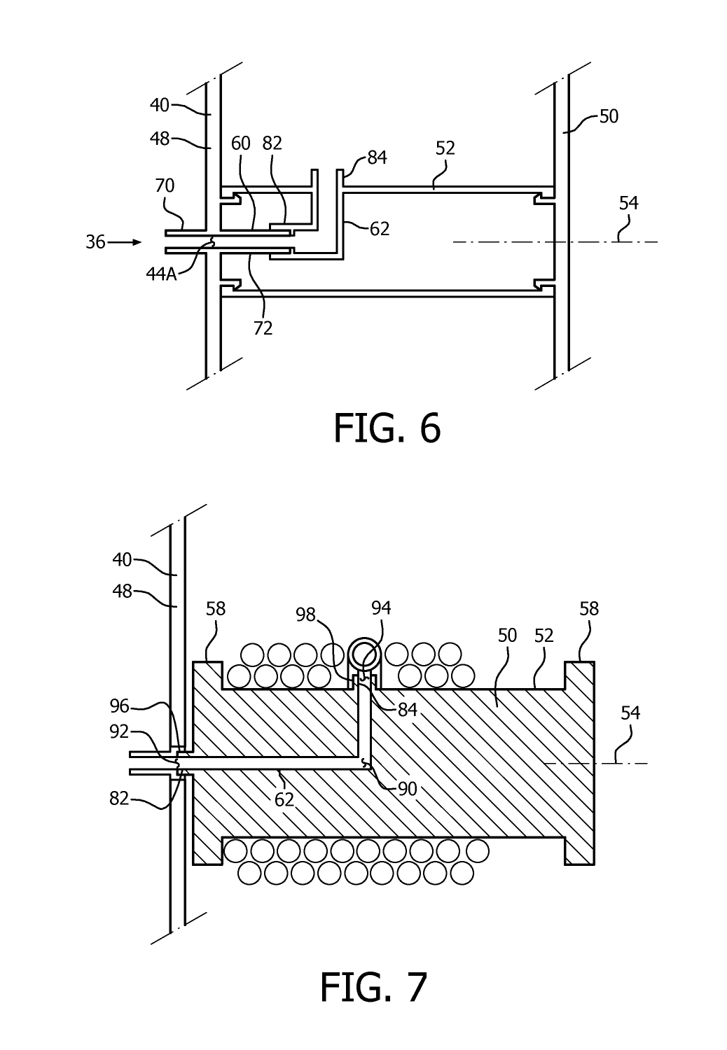

In another embodiment, shown in FIG. 6, stationary first conduit 60 is unitary with housing assembly sidewall assembly 40. That is, housing assembly sidewall assembly 40 is molded to form a passage through one of first or second planar members 48, 49 at a location corresponding to spool body axis of rotation 54. A passage includes a first inlet opening 44A through housing assembly sidewall assembly 40 as well as a first circular collar 70, disposed about first inlet opening 44A extending outwardly from housing assembly sidewall assembly 40, and a second circular collar 72, disposed about first inlet opening 44A extending inwardly from housing assembly sidewall assembly 40. Second circular collar 72 may also act as an axle for spool body 50.

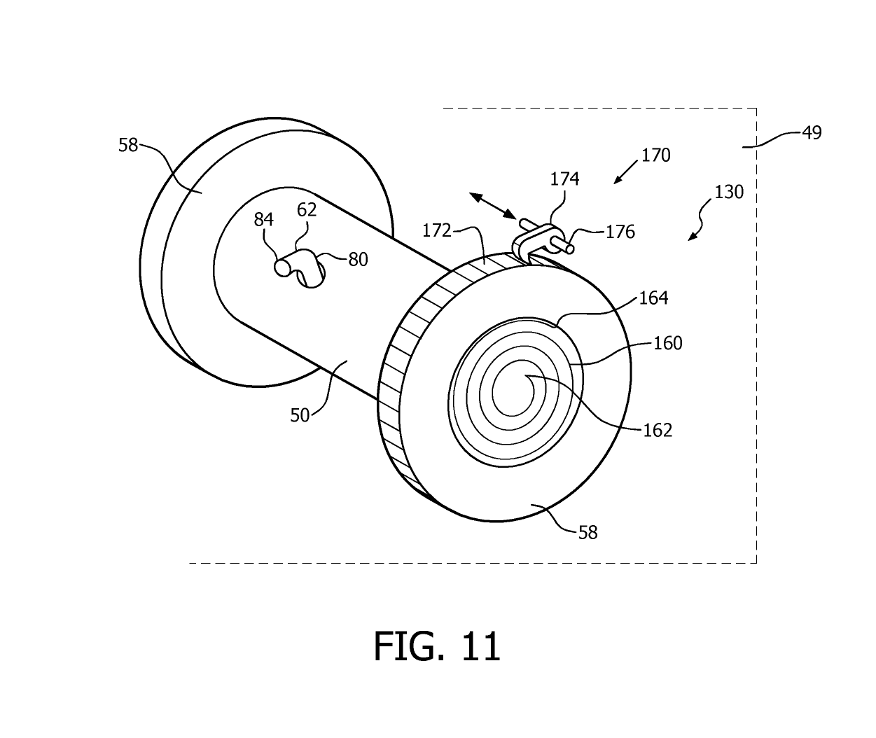

Movable second conduit 62 may be unitary (FIG. 6) with spool body 50 or be a separate body 80 (FIG. 5). In either configuration, movable second conduit 62 has a first end 82 and a second end 84. Movable second conduit first end 82 is an inlet that is structured to be coupled to, and in fluid communication with, stationary first conduit second end 68. Movable second conduit second end 84 is structured to be coupled to, and in fluid communication with, second hose 22. Movable second conduit 62 is a bent tubular member. That is, movable second conduit 62 includes at least one bend and preferably two. Movable second conduit first end 82 is generally straight and is disposed along spool body axis of rotation 54. The medial portion of movable second conduit 62 bends about ninety degrees, so as to extend radially relative to spool body 50. Movable second conduit second end 84 extends through spool body outer surface 52 and is exposed. Movable second conduit body second end 84 may include an additional bend so that the axis of the movable second conduit body second end 84 extends generally tangent to spool body outer surface 52 (FIG. 11).

In another embodiment, shown in FIG. 7, movable second conduit 62 is a passage 90 extending through spool body 50. That is, spool body 50 may be substantially solid, or solid in the portion forming movable second conduit 62 and passage 90 may be cut therethrough. Alternately, the entire spool body 50 may be substantially hollow and define passage 90 (not shown). In this embodiment, movable second conduit first end 82 is a passage inlet 92 disposed on an axial end of spool body 50 at spool body axis of rotation 54, and, movable second conduit body second end 84 is a passage outlet 94 disposed on spool body outer surface 52. Movable second conduit inlet 92 and outlet 94 may each include a collar 96, 98 which in one exemplary embodiment is circular. Movable second conduit outlet collar 98 may extend generally tangentially to spool body outer surface 52.

Hose management system 30 is configured as follows. Spool body 50 is disposed within housing assembly enclosed space 42. Spool body 50 is rotatably coupled to housing assembly 32 and structured to rotate about spool body axis of rotation 54. For example, if spool body 50 includes axial cavities 57 and the inner surface of housing assembly sidewall assembly 40 includes aligned circular projections 59, spool body 50 is disposed between the two projections 59 with projections 59 disposed in axial cavities 57. Rotatable coupling stationary first conduit 60 extends through housing assembly sidewall assembly 40, such as through one of housing assembly sidewall assembly planar members 48. 49, and is disposed substantially along spool body axis of rotation 54. Movable second conduit 62 is disposed with, or as noted above, is unitary with, spool body 50. Second conduit body second end 84 extends over spool body outer surface 52 and, extends generally tangent thereto. First conduit body second end 68 is coupled to, and in fluid communication with, second conduit body first end 82. Moreover, second conduit body 80 is rotatably coupled to first conduit body 64. Thus, second conduit body 80 may rotate along with spool body 50.

In this configuration, passage 19, i.e. first hose 20, is coupled to, and is in fluid communication with, both pressure generating system 12 and first conduit body first end 66. The present invention contemplates that first hose 20 is of a limited length. That is, first hose 20 may have a sufficient length to extend from a floor to the top of a nightstand. Thus, pressure generating system 12 may be placed on the floor and hose management system 30 may be placed on the nightstand with a minimal length of first hose 20 being visible. Alternatively, first hose 20 may be very short, e.g. just a few inches, wherein first hose 20 merely provides a connection between pressure generating system 12 and hose management system 30.

Second hose 22 is coupled to, and is in fluid communication with, both second conduit body second end 84 and patient interface 16. Thus, there is fluid communication from pressure generating system 12 to patient interface 16. Second hose 22 is selectively wrapped about spool body 50. That is, second hose 22 is structured to move between two configurations, a first configuration, wherein second hose 22 is substantially disposed within housing assembly 32, and a second configuration, wherein at least a portion of second hose 22 extends from housing assembly 32. That is, when system 10 adapted to provide a regimen of respiratory therapy is not in use, second hose 22 may be in the first configuration wherein second hose 22 is substantially, or entirely, disposed within housing assembly enclosed space 42. In this configuration, second hose 22 is not visible, thus accomplishing the objective of conduit assembly 14 being of limited visibility and, in an exemplary embodiment, substantially concealed.

When a user desires to utilize system 10, the user pulls a length of second hose 22 from housing assembly 32. That is, the user unwinds a portion, or all, of second hose 22 as needed from spool body 50. The user may, for example, utilize system 10 while the user sleeps, the length of second hose 22 allowing the user to shift while sleeping. When the user no longer desires to use system 10, the exposed portion of second hose 22 is wound about spool body 50. That is, the exposed portion of second hose 22 is returned to enclosed space 42.

Hose management system 30 described above may include additional features such as a pocket 100 (FIG. 2) for patient interface 16 and a retraction device 130. That is, as described above, patient interface 16 is disposed at the end of second hose 22. As further described above, housing assembly sidewall assembly openings 44 are, about the same size as hoses 20, 22. In this configuration, patient interface 16 cannot pass through opening 44 into enclosed space 42. Thus, patient interface 16 is disposed on the outside of housing assembly 32. There are at least two problems with this configuration. First, patient interface 16 is exposed and may be damaged or contaminated. Second, patient interface 16 also has the undesirable appearance of medical equipment. The problems may be addressed by providing a covered pocket 100 for the patient interface 16.

That is, as shown in FIG. 2, a portion of the depending sidewall 47, as well as a portion of the planar members 48, 49, may be made movable. This movable portion of the housing assembly sidewall assembly 40 is a cover 102. Further, an internal wall 104 may be positioned under cover 102. Internal wall 104 extends between sidewall assembly planar members 48, 49. The present invention contemplates that internal wall 104 includes a passage 44C sized to correspond to the size of second hose 22. Internal wall 104 is set inwardly from the perimeter of sidewall assembly planar members 48, 49. Thus, internal wall 104 defines the bottom of covered pocket 100. Further, internal wall 104 includes a shaped cavity 106, cavity 106 being shaped to correspond to the shape of patient interface 16. Thus, internal wall 104 may act as a seat for patient interface 16.

The present invention contemplates that cover 102 is hinged to housing assembly sidewall assembly 40. In this configuration, cover 102 is structured to be selectively moved between two positions; a first position, wherein cover 102 is disposed over and adjacent pocket 100, i.e. over and adjacent internal wall 104, and a second position, wherein cover 102 is pivoted away from internal wall 104 thereby exposing internal wall 104. Internal wall 104 may include a seat 107 structured to conform to the shape of patient interface 16. Cover 102 may include a latch such as, but not limited to, a ball-and-detent, structured to maintain the cover in the first configuration. Further, second hose 22 extends through passage 44C in internal wall 104 with the exposed end being coupled to patient interface 16. Thus, when second hose 22 is in the first configuration, second hose 22 is entirely, or substantially, disposed within housing assembly enclosed space 42 wherein second hose 22 cannot be seen. Further, when second hose 22 is in the first configuration and cover 102 is in its first configuration, patient interface 16 is disposed within pocket 100 and covered. Thus, patient interface 16 is also not visible when not in use.

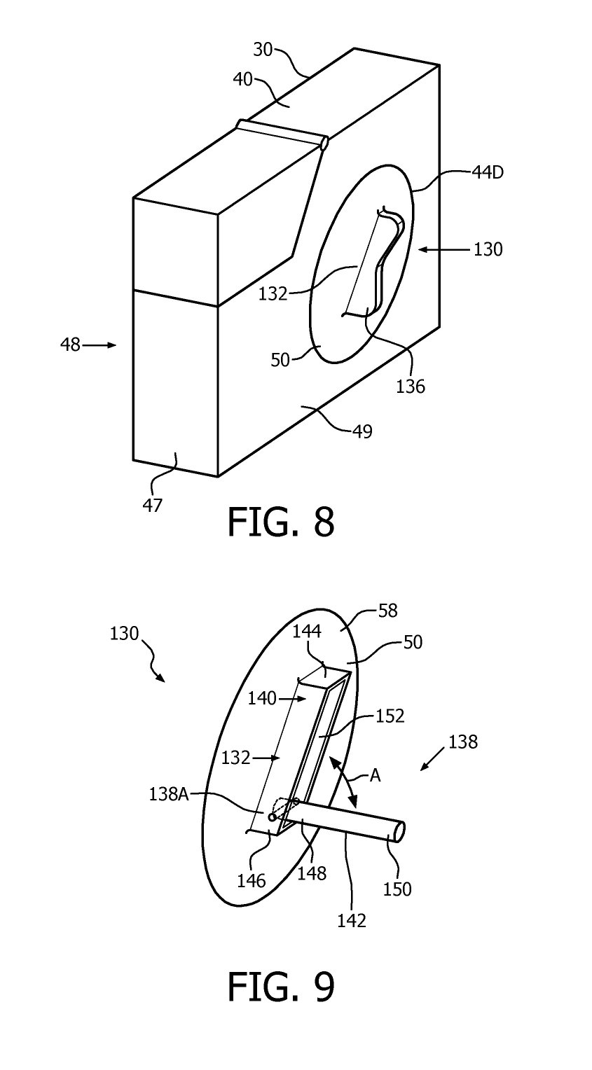

The present invention contemplates that spool assembly 34 includes a retraction device 130, as shown in FIG. 8. Retraction device 130 is structured to rotate spool body 50 so that, if second hose 22 is coupled to rotatable coupling 36 as described above, second hose 22 is wound about spool body 50 as spool body 50 rotates. In exemplary embodiment, retraction device 130 is substantially automatic, as described below, but retraction device 130 may be manual as well. For example, spool body 50 may include an axial crank extension 132. That is, spool body 50 may include a member extending axially from spool body 50 opposite rotatable coupling 36. Axial crank extension 132 may be coupled to, fixed to, or be unitary with spool body 50. Crank extension 132 may be, shaped so as to be gripped by a user. For example, as shown crank extension 132 is a planar crank member 136 wherein the plane of planar crank member 136 is substantially aligned with spool body axis of rotation 54.

In another embodiment, shown in FIG. 9, crank extension 132 may be a L-shaped member 138 or a foldable L-shaped member 138A. That is, L-shaped member 138 has an elongated first member 140 and an elongated second member 142. First member 140 may be unitary with spool body 50. Each L-shaped member first and second members 140, 142 have a first end 144, 148 and a second end 146, 150, respectively. L-shaped member first member 140 may include a longitudinally extending cavity 152. L-shaped member first member first end 144 may be coupled to axial crank extension 132, but is unitary therewith in an exemplary embodiment. L-shaped member second member first end 148 is pivotally coupled to L-shaped member first member second end 146. Thus, L-shaped member second member 142 may move, as indicated by arrow A between two positions, a collapsed first position, wherein L-shaped member second member 142 is at least partially disposed in L-shaped member first member cavity 152, and an extended second position, wherein L-shaped member second member 142 extends generally perpendicular to L-shaped member first member 140.

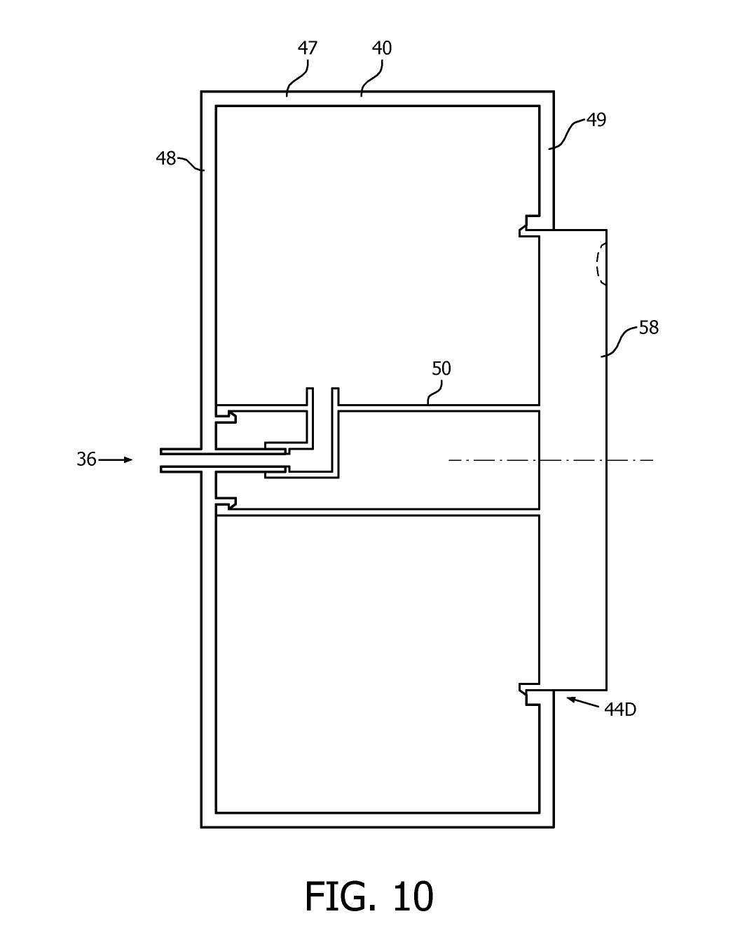

Alternately, manual retraction device 130 may utilize spool body hub 58 as an actuator, as shown in FIG. 10. That is, in this embodiment, spool body hub 58 extends through housing assembly sidewall assembly 40. Housing assembly sidewall assembly 40 includes a larger opening 44D through which spool body hub 58 may extend. The present invention contemplates that spool body hub 58 corresponds substantially to the size of larger opening 44D so that an observer cannot see into housing assembly enclosed space 42. Exposed spool body hub 58 may include a finger detent, or similar construct, structured to enable the user to better rotate exposed hub 58 and therefore spool body 50.

In the exemplary embodiment, however, retraction device 130 is substantially automatic. For example, as shown in FIG. 11, retraction device 130 may include a spiral torsion spring 160 structured to cause spool body 50 to rotate relative to housing assembly 32. Torsion spring 160 has a first end 162 and a second end 164. Torsion spring first end 162 is coupled to housing assembly 32 and torsion spring second end 164 is coupled to spool body 50. Torsion spring 160 is not, or substantially not, under tension when second hose 22 is in the first configuration; that is, when second hose 22 is wound about spool body 50. Thus, when a user moves second hose 22 to the second configuration, i.e. when a user pulls a portion of second hose 22 from housing assembly enclosed space 42, torsion spring 160 is placed under tension and biases spool body 50 to second hose 22 first configuration. That is, the tension in torsion spring 160 causes spool assembly 34 to wind second hose 22 about spool body 50. Put another way, spiral torsion spring 160 moves between a first configuration, wherein there is an insufficient spring force to cause spool body 50 to rotate, and a second configuration, wherein there is a sufficient spring force to cause spool body 50 to rotate.

To prevent retraction device 130 from constantly drawing second hose 22 into housing assembly enclosed space 42, retraction device 130 includes a ratchet gear and pawl assembly 170. Ratchet gear and pawl assembly 170 is structured to maintain torsion spring 160 a selected configuration. Ratchet gear and pawl assembly 170 includes a ratchet gear 172 and a movable pawl 174. The present invention contemplates that ratchet gear 172 is disposed on spool body 50. For example, ratchet gear 172 may be disposed inside hollow spool body 50, or, about the radial surface of spool hub 58. Pawl 174 is disposed adjacent ratchet gear 172 and is structured to selectively engage ratchet gear 172. That is, ratchet gear 172 may rotate in one direction with pawl 174 moving over ratchet gear 172 teeth. If ratchet gear 172 rotates in the opposite direction, pawl 174 engages ratchet gear 172 teeth and prevents rotation of ratchet gear 172. In an exemplary embodiment, pawl 174 is structured to selectively engage ratchet gear 172 until disengaged. For example, pawl 174 may be disposed on a rod 176 extending generally parallel to spool axis of rotation 54. Pawl 174 on rod 176 may be moved between a first position, wherein pawl 174 selectively engages ratchet gear 172, and a second position, wherein pawl 174 does not engage ratchet gear 172.

In this configuration, retraction device 130 operates as follows. When second hose 22 is in the first configuration, spiral torsion spring 160 is in its first configuration. Further, pawl 174 is engaging ratchet gear 172. When the user pulls second hose 22 from housing assembly enclosed space 42, i.e. when second hose 22 is moved to the second configuration, spool body 50 spins causing torsion spring 160 to be in torsion spring 160 second configuration. Pawl 174, however, engages ratchet gear 172 thereby preventing the bias of torsion spring 160 from causing spool body 50 to move. When the user ceases to use system 10, the user moves pawl 174 to pawl 174 second position, thereby releasing spool body 50. Once spool body 50 is no longer held in a specific position, the bias of torsion spring 160 causes spool body 50 to move which in turn winds second hose 22 about spool body 50. That is, the bias of torsion spring 160 returns second hose 22 to the first configuration.

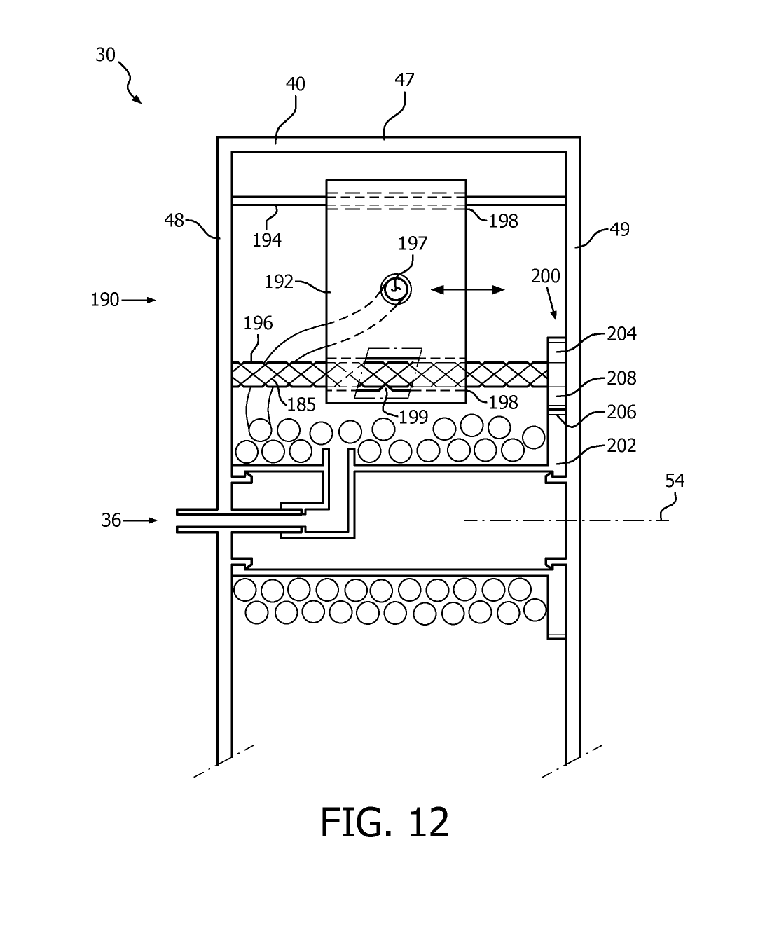

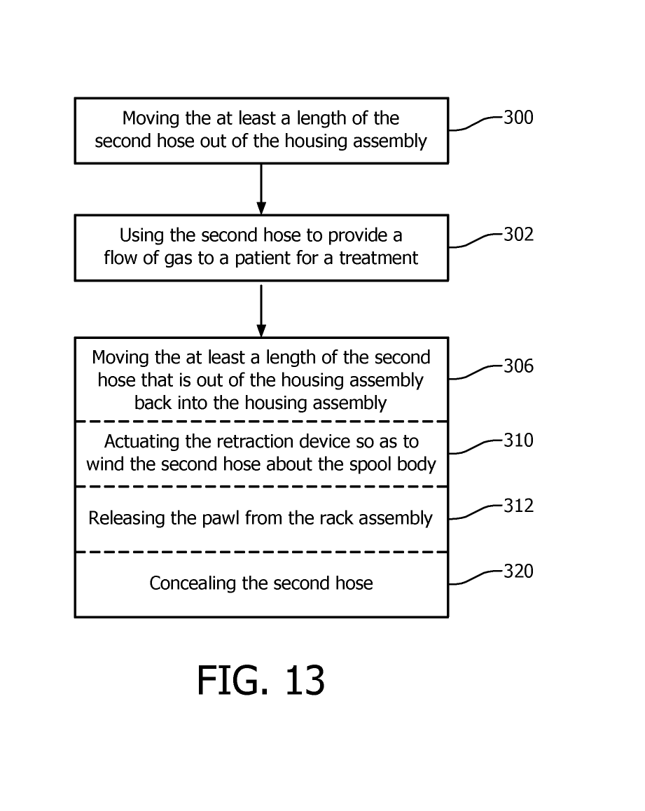

If spool body 50 is elongated, it may be desirable to direct the winding of second hose 22. That is, it is not desirable to have second hose 22 wrapped about a narrow portion of spool body 50. Thus, as shown in FIG. 12, housing assembly 32 may include a hose guide device 190 structured to cause second hose 22, as it is being wound about spool body 50, to reciprocate relative to the axis of rotation of spool body 50. The present invention contemplates that hose guide device 190 includes guide housing 192, a guide rod 194 and a drive rod 196. Guide rod 194 and drive rod 196 are disposed within housing assembly enclosed space 42 and extend in a spaced, parallel relation. Moreover, guide rod 194 and drive rod 196 are substantially parallel to the spool body axis of rotation 54. Drive rod 196 is rotatably coupled to housing assembly 32. Drive rod 196 includes a bi-directional groove 185 thereon. In one exemplary embodiment, guide housing 192 is a planar body defining a normal opening 197, sized to accommodate second hose 22, and two planar passages 198. Planar passages 198 are sized to accommodate guide rod 194 and drive rod 196. In one planar passage 198, through which drive rod 196 extends, as discussed below, there is a tooth 199.

Hose guide device 190 includes a drive device 200. Drive device 200 has a first component 202 and a second component 204. Drive device first component 202 is coupled to, and fixed to, spool body 50. Drive device second component 204 is coupled to, and fixed to, drive rod 196. For example, the outer radial surface of spool hub 58 may include teeth 206, which are drive device first component 202. Drive rod 196 may include a gear 208 fixed thereto and structured to engage or mesh with spool hub teeth 206. Thus, when spool body 50 is rotated, the rotational motion is transferred to drive rod 196. Alternative drive device 200 includes, but is not limited to, a belt drive (not shown).

Guide housing 192 is movably coupled to guide rod 194 and drive rod 196 which each extend through different housing body planar passages 198. Drive rod 196 extends through planar passage 198 having a tooth 199. The tooth 199 is disposed in bi-directional groove 185. In this configuration, the rotation of drive rod 196 causes tooth 199 to engage bi-directional groove 185 which, in turn, causes guide housing 192 to move along drive rod 196. When tooth 199 reaches one end of bi-directional groove 185, i.e. where groove 185 ends adjacent an end of drive rod 196, guide housing 192 will reverse its direction due to the nature of bi-directional groove 185. Thus, in this configuration, guide housing 192 reciprocates over drive rod 196. Moreover, second hose 22 is passed through guide housing normal opening 197. Thus, as guide housing 192 reciprocates over drive rod 196, second hose 22 is moved axially relative to spool body 50. As spool body 50 is rotating, second hose 22 is distributed substantially evenly over spool body 50.

Thus, when using system 10 adapted to provide a regimen of respiratory therapy with hose management system 30 as described above, the user performs the following steps: moving 300 at least a length of second hose 22 out of housing assembly 32, using 302 second hose 22 to provide a flow of gas to a patient for a treatment, and, after the patient has completed the treatment, moving 306 the at least a length of second hose 22 that is out of housing assembly 32 back into housing assembly 32. If hose management system 30 includes retraction device 130, as described above, the step of moving 306 the at least a length of second hose 22 that is out of housing assembly 32 back into housing assembly 32 includes the step of actuating 310 retraction device 130 so as to wind second hose 22 about spool body 50. More specifically, if retraction device 130 includes torsion spring 160, ratchet gear 162 and pawl 164, as described above, the step of moving 306 the at least a length of second hose 22 that is out of housing assembly 32 back into housing assembly 32 includes the step of releasing 312 pawl 14 from ratchet gear assembly 172 thereby releasing spiral torsion spring 160 and causing spool body 50 to rotate and wind second hose 22 about spool body 50.

The present invention contemplates that housing assembly 32 is shell 46 as defined above. Thus, the step of moving 306 the at least a length of second hose 22 that is out of housing assembly 32 back into housing assembly 32 further includes the step of substantially concealing 320 second hose.

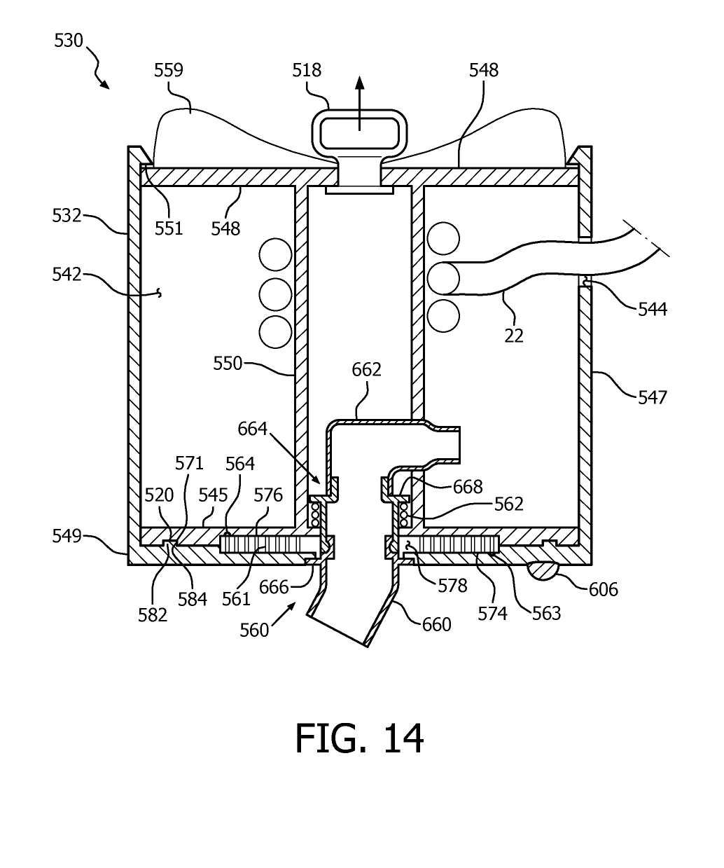

Another embodiment is shown in FIG. 14. In this embodiment, hose management system 530 includes a housing assembly 532 having a top, first sidewall planar member 548, and a bottom, second sidewall planar member 549. First and second sidewall planar members 548, 549 have a generally circular shape. First sidewall planar member 548 is a unitary body with a spool body 550. Spool body 550 is also unitary with an inner plate 545. First sidewall planar member 548 includes a projection 559 that is shaped for use as a winding handle. First sidewall planar member 548 also includes a handle 518 that is rotatably coupled to first sidewall planar member 548 and disposed along spool body 550 axis of rotation.

Second sidewall planar member 549 includes a depending sidewall 547 that extends toward first sidewall planar member 548. Depending sidewall 547 is slightly longer than the combined length of spool body 550 and the thickness of first sidewall planar member 548. Depending sidewall 547 also includes a latching edge 551 extending inwardly near the distal end of depending sidewall 547. Depending sidewall 547 includes an outlet opening 544 for the second hose 22. Depending sidewall 547 and second sidewall planar member 549 substantially define the enclosed space 542.

In this configuration, first sidewall planar member 548, as well as spool body 550 and inner plate 545 may be disposed in housing assembly enclosed space 542. As first sidewall planar member 548 is coupled to second sidewall planar member 549 by the latching edge 551, first sidewall planar member 548 and second sidewall planar member 549 are rotatably coupled to each other. Moreover, because depending sidewall 547 has a greater length than the combined length of spool body 550 and the thickness of first sidewall planar member 548, the first sidewall planar member 548, as well as spool body 550 and inner plate 545 may shift, i.e. move in a direction parallel to the axis of rotation of spool body 550, relative to second sidewall planar member 549. This allows for the operation of a spool assembly retraction device 560 described below.

Spool assembly retraction device 560 includes a torsion spring 561, a compression spring 562 as well as a protruding, first circular rack 570 and shaped cavities 572, 574, 576 on the inner surfaces of second sidewall planar member 549 and inner plate 545. That is, circular rack 570 and shaped cavities 570, 572, 574, 576 are disposed on the surfaces of second sidewall planar member 549 and inner plate 545 that face each other. Shaped cavity 574 is disposed on the upper surface of second sidewall planar member 549. Shaped cavity 574 is generally circular. Shaped cavity 576 is disposed on the lower surface of inner plate 545. Shaped cavity 576 is also generally circular and is sized to correspond to shaped cavity 574. Thus, when second sidewall planar member 549 and inner plate 545 are disposed adjacent to each other, shaped cavities 574, 576 form a spring cavity 578. Torsion spring 561 is disposed in spring cavity 578. Torsion spring 561 has a first end 563 and a second end 564. Torsion spring first end 563 is coupled to second sidewall planar member 549 and torsion spring second end 564 is coupled to inner plate 545. Thus, the second sidewall planar member 549 and inner plate 545 rotate relative to each other and are biased by torsion spring 561 as described above. That is, torsion spring 561 acts as a spring-loaded coiler.

First circular rack 570 extends from the upper surface of second sidewall planar member 549. First circular rack 570 extends over a substantially circular path and includes upwardly extending teeth 582, i.e. axially inwardly relative to the spool body 550 axis of rotation. Shaped cavity 572 also extends over a substantially circular path sized to substantially correspond to first circular rack 570. When second sidewall planar member 549 and inner plate 545 are disposed adjacent to each other, first circular rack 570 is disposed within shaped cavity 572. Shaped cavity 572 includes a second circular rack 571 having downwardly extending teeth 584, i.e. axially outwardly relative to the spool body 550 axis of rotation. Thus, when second sidewall planar member 549 and inner plate 545 are disposed adjacent to each other, i.e. when first circular rack 570 is disposed in shaped cavity 572, first circular rack teeth 582 engage second circular rack teeth 584. This engagement of first circular rack teeth 582 and second circular rack teeth 584 prevent the second sidewall planar member 549 and inner plate 545 from rotating relative to each other. It is noted, first circular rack teeth 582 and second circular rack teeth 584 may be angled so that rotation in only one direction is prevented. The compression spring 562 is structured to bias second sidewall planar member 549 and inner plate 545 toward each other.

As noted above, first sidewall planar member 548, as well as spool body 550 and inner plate 545 may be disposed in housing assembly enclosed space 542 and may move axially relative to second sidewall planar member 549. This motion is accommodated by a sliding rotatable coupling 536. That is, sliding rotatable coupling 536 is, essentially, similar to the hose management system rotatable coupling 36 described above, except that sliding rotatable coupling 536 allows a first sliding conduit 660 to be slidably, and rotatably, coupled to a second sliding conduit 662. That is, there is an axially slidable interface 664 between elements of sliding rotatable coupling 536. Further, first sliding conduit 660 includes two radially extending flanges 666 and 668. An outer flange 666 is coupled to, and engages the second sidewall planar member 549. An inner flange 668 is disposed within spool body 550. Compression spring 562 extends between inner flange 668 and the inner surface of inner plate 545. In this configuration, compression spring 562 biases inner plate 545 and second sidewall planar member 549 together.

Spool assembly retraction device 560 operates as follows. A user grips second sidewall planar member 549 and holds it, thereby making second sidewall planar member 549 essentially stationary. The user then utilizes handle 518 to lift first sidewall planar member 548, as well as spool body 550 and inner plate 545, away from second sidewall planar member 549. At this time, first sliding conduit 660 and second sliding conduit 662 also move axially relative to each other, but do not disengage. That is, the sliding rotatable coupling 536 still defines a passage. This action further moves first circular rack 570 out of shaped cavity 572, thus out of engagement with second circular rack 571. At this point first sidewall planar member 548, as well as spool body 550 and inner plate 545, may rotate relative to second sidewall planar member 549. The user may simply pull on second conduit 22, or, may use projection 559 to move first sidewall planar member 548 thereby extending second conduit 22 from enclosed space 542. This action also causes first sidewall planar member 548, spool body 550 and inner plate 545 to rotate relative to second sidewall planar member 549 which in turn creates a biasing force in the torsion spring 561.

After use is completed, the user simply grips second sidewall planar member 549 and holds it while lifting handle 518. Again, this action moves first sidewall planar member 548, as well as spool body 550 and inner plate 545, away from second sidewall planar member 549 and moves first circular rack 570 out of shaped cavity 572. As before, when first circular rack 570 is moved out of shaped cavity 572, the first and second circular racks 570, 571 are no longer engaged. At this point first sidewall planar member 548, as well as spool body 550 and inner plate 545, may rotate relative to second sidewall planar member 549. The bias in the torsion spring 561 causes first sidewall planar member 548, as well as spool body 550 and inner plate 545, to rotate relative to second sidewall planar member 549, thereby winding second conduit 22 within the housing assembly 532.

As also shown in FIG. 14, the housing assembly 532 may include an extending lug 600. Lug 600 is sized and shaped to fit snugly within a cavity (not shown) on pressure generating system 12. Thus, the user may temporarily couple the housing assembly 532 to pressure generating system 12. Such a configuration allows the pressure generating system 12 and hose management system 30 to be arranged in an orderly manner. Further, having hose management system 30 temporarily coupled to pressure generating system 12 may allow selected operations, e.g. the winding of second conduit 22 to be accomplished more easily.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by on and the same item of hardware. The word “a” or “an” preceding an element does not exclude the presence of a plurality of such elements. In any device claim enumerating several means, several of these means may be embodied by one and the same item of hardware The mere fact that certain elements are recited in mutually different dependent claims does not indicate that these elements cannot be used in combination.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

1. A hose management system for use with a system adapted to provide a regimen of respiratory therapy for a patient, including a conduit assembly having at least an elongated, flexible second hose, the hose management system comprising:
a housing assembly having a sidewall assembly defining a substantially enclosed space;
a rotatable spool assembly having a spool body with a generally cylindrical outer surface, wherein the spool body is disposed in the housing assembly enclosed space and rotatably coupled to the housing assembly; and
a rotatable coupling defining a passage, wherein the rotatable coupling includes a rotatable conduit extending through the housing assembly sidewall and the spool body, and wherein the second hose is substantially wrapped about the spool body and substantially disposed within the housing assembly enclosed space, and wherein the housing assembly sidewall assembly defines a shell that substantially conceals any object in the housing assembly enclosed space.

2. (canceled)

3. The hose management system of claim 2, wherein the housing assembly sidewall assembly includes three or less openings, each opening being sized to be substantially the same size as the second hose.

4. The hose management system of claim 1, wherein the housing assembly sidewall assembly includes a first substantially planar member and a second substantially planar member, the first and second planar members disposed substantially parallel to each other, and wherein the spool body is rotatably coupled to, and extending between, the first and second planar members.

5. The hose management system of claim 1, wherein:
the housing assembly includes a cover;
the housing assembly sidewall assembly includes an internal wall, the internal wall defining a pocket having an opening therethrough;
the pocket being a selectably enclosed space separated by the internal wall from the housing assembly sidewall assembly enclosed space and
the cover being a sidewall movably coupled to the housing assembly sidewall assembly, the cover being moveable between a first position, wherein the cover is disposed substantially over and adjacent to the internal sidewall, and a second position, wherein the cover is substantially spaced from the internal sidewall.

6. The hose management system of claim 5, wherein the internal sidewall includes a shaped seat, the seat being shaped to correspond to the shape of the patient interface.

7. The hose management system of claim 1, wherein:
the rotatable coupling defining a passage includes a stationary first conduit and a second movable conduit;
the rotatable coupling first conduit coupled to, and extending through the housing assembly sidewall assembly, the first conduit having a body with a first end and a second end;
the rotatable coupling second conduit having a body with a first end and a second end, the rotatable coupling second conduit disposed within the spool body and the rotatable coupling second conduit second end disposed at the spool body cylindrical outer surface;
the rotatable coupling first conduit second end coupled to, and in fluid communication with, the rotatable coupling second conduit first end; and
wherein the rotatable coupling second conduit second end is structured to be coupled to the second hose; and
whereby the second hose may be wound about the spool body and disposed within the housing assembly enclosed space.

8. The hose management system of claim 7, wherein:
the rotatable coupling first conduit is a first, substantially straight tubular body, the first tubular body extending through the housing assembly sidewall assembly;
the rotatable coupling second conduit is a bent tubular body; the spool body being hollow; and
the rotatable coupling second conduit body being partially disposed within the spool body with the rotatable coupling second conduit second end disposed at the outer surface of the spool body.

9. The hose management system of claim 7, wherein:
the rotatable coupling first conduit is unitary with the housing assembly sidewall assembly, the rotatable coupling first conduit having a first circular collar and a second circular collar;
the first circular collar extending outwardly from the housing assembly sidewall assembly;
the second circular collar extending inwardly from the housing assembly sidewall assembly into the enclosed space;
the rotatable coupling second conduit body is a bent tubular member; the spool body being hollow; and
the rotatable coupling second conduit tubular member being partially disposed within the spool body with the rotatable coupling second conduit second end disposed at the outer surface of the spool body.

10. The hose management system of claim 7, wherein:
the rotatable coupling first conduit is unitary with the housing assembly sidewall assembly, the rotatable coupling first conduit having a first circular collar and a second circular collar;
the first circular collar extending outwardly from the housing assembly sidewall assembly;
the second circular collar extending inwardly from the housing assembly sidewall assembly into the enclosed space;
the spool body defining a passage, the spool passage being the rotatable coupling second conduit;
the rotatable coupling second conduit first end being an inlet disposed substantially on the axis of rotation of the spool body; and
the rotatable coupling second conduit first end being a collar disposed on the outer surface of the spool body.

11. The hose management system of claim 1, wherein the spool assembly includes a retraction device, the retraction device structured to rotate the spool body so that, if the second hose is coupled to the rotatable coupling, the second hose is wound about the spool body.

12. The hose management system of claim 11, wherein:
the refraction device includes a spiral torsion spring having a first end and a second end;
the spiral torsion spring first end being coupled to the housing assembly; the spiral torsion spring second end being coupled to the spool body; whereby the spiral torsion spring moves between a first configuration, wherein there is an insufficient spring force to cause the spool body to rotate, and a second configuration, wherein there is a sufficient spring force to cause the spool body to rotate; and
whereby the second hose may be coupled to, and in fluid communication with, the rotatable coupling so that when the second hose is wound about the spool body, the spiral torsion spring is in the first configuration and when the second hose is unwound from the spool body the torsion spring is in the second configuration.

13. The hose management system of claim 11, wherein the retraction device includes a ratchet gear and pawl assembly, the ratchet gear and pawl assembly structured to maintain the torsion spring a selected configuration.

14. The hose management system of claim 1, wherein the housing assembly includes a hose guide device, the hose guide device disposed adjacent the spool body and structured to cause a hose being wound about the spool body to reciprocate relative to the axis of rotation of the spool body.

15. The hose management system of claim 14, wherein:
the hose guide device includes guide housing, a guide rod and a drive rod;
the guide housing is a body defining a passage sized to accommodate the second hose and a tooth;
the guide rod disposed within the housing assembly sidewall assembly enclosed space and extending substantially parallel to the spool body axis of rotation;
the drive rod having drive device first component and a bi-directional groove thereon, the drive rod disposed within the housing assembly sidewall assembly enclosed space and extending substantially parallel to the spool body axis of rotation;
the spool having a drive device second component;
the drive device first and second components engaging each other;
the guide housing movably coupled to the guide rod and the drive rod with the guide housing tooth disposed in the drive rod groove; and
whereby, as the spool body rotates, the drive device causes the drive rod to rotate and the guide housing to reciprocate along the drive rod.

16. A method of using a hose management system, wherein the hose management system is used in conjunction with a system adapted to provide a regimen of respiratory therapy for a patient, the system including: a pressure generating system adapted to produce a flow of gas, a conduit assembly, and a patient interface, the conduit assembly disposed between and in fluid communication with both the pressure generating system and the patient interface, the conduit assembly including at least a first passage and an elongated, flexible second hose, the hose management system including a housing assembly having a sidewall assembly defining a substantially enclosed space, a rotatable spool assembly having a spool body with a generally cylindrical outer surface, the spool body disposed in the housing assembly enclosed space and rotatably coupled to the housing assembly, a rotatable coupling defining a passage and having a first upstream end and a second downstream end, the rotatable coupling being a rotatable conduit extending through the housing assembly sidewall and spool body, the first passage coupled to, and in fluid communication with, the rotatable coupling first end, and the second hose coupled to the rotatable coupling second end, wherein the conduit assembly second hose may be substantially wrapped about the spool body and substantially disposed within the housing assembly enclosed space, the method comprising the steps of:
moving at least a length of the second hose out of the housing assembly; using the second hose to provide a flow of gas to a patient for a treatment; and
after the patient has completed the treatment, moving the at least a length of the second hose that is out of the housing assembly back into the housing assembly; and wherein the housing assembly sidewall assembly substantially conceals any object in the housing assembly enclosed space.

17. The method of claim 16, wherein, the spool assembly includes a retraction device, the retraction device structured to rotate the spool body and wherein the step of moving the at least a length of the second hose that is out of the housing assembly back into the housing assembly includes the step of actuating the retraction device so as to wind the second hose about the spool body.

18. The method of claim 17, wherein the retraction device includes a spiral torsion spring having a first end and a second end, the spiral torsion spring first end being coupled to the housing assembly, the spiral torsion spring second end being coupled to the spool body, whereby the spiral torsion spring moves between a first configuration, wherein there is an insufficient spring force to cause the spool body to rotate, and a second configuration, wherein there is a sufficient spring force to cause the spool body to rotate, the spiral torsion spring being in the second configuration when a length of the second hose has been moved out of the housing assembly, the retraction device further includes a ratchet gear and pawl assembly, the ratchet gear and pawl assembly structured to maintain the spool body a selected configuration and wherein the step of actuating the retraction device so as to wind the second hose about the spool body includes the step of releasing the pawl from the ratchet gear assembly thereby releasing the spiral torsion spring and causing the spool body to rotate and wind the second hose about the spool body.

19. The method of claim 16, wherein the housing assembly defines a shell that substantially conceals any object in the housing assembly enclosed space and wherein the step of moving the at least a length of the second hose that is out of the housing assembly back into the housing assembly includes the step of substantially concealing the second hose.
